(19) United States
(12) Patent Application Publication
(10) Pub. No.: US 2024/0115319 A1
(43) Pub. Date: Apr. 11, 2024

(54) LASER RESECTION DEVICE

(71) Applicant: CYCLONE BIOSCIENCES, LLC, Phoenix, AZ (US)

(72) Inventor: Stephen E. Griffin, Peoria, AZ (US)

(73) Assignee: CYCLONE BIOSCIENCES, LLC, Phoenix, AZ (US)

(21) Appl. No.: 18/479,496

(22) Filed: Oct. 2, 2023

Related U.S. Application Data
(60) Provisional application No. 63/413,704, filed on Oct. 6, 2022.

(51) Int. Cl. A61B 18/24

(52) U.S. Cl. CPC A61B 18/245; A61B 2018/2205

(57) ABSTRACT

An all-optical-fiber device configured to supply, to a target, laser radiation having characteristics sufficient for ablating a biological tissue in a manner that is consistent and competitive with the tissue removal geometry and volume provided by conventional electrocautery resection devices. The desired effect is achieved by configuring a facet of the optical fiber or an internal surface of the fiber's termination cap as a surface providing total-internal-reflection of light substantially at every point of such surface while having normals drawn to immediately adjoining surface portions be not parallel with respect to one another.

### CROSS-REFERENCE TO RELATED APPLICATIONS

This US patent application claims priority from and benefit of the U.S. Provisional Patent Application No. 63/413,704 filed on Oct. 6, 2022. The disclosure of this provisional application is incorporated by reference herein.

### TECHNICAL FIELD

The present invention relates to an optical-fiber-based all-optical accessory for a medical device configured to cut, ablate, resect, vaporize, coagulate a tissue with the use of light, as well as to a medical device employing such accessory and, in particular, to the accessory including a side-fire (interchangeably referred to herein as radially-emitting) optical fiber element.

### RELATED ART

Resection of obstructive tissues—for example, to achieve the relief of lower urinary tract symptoms (referred to in the art as LUTS) caused by enlarged prostate glands—has been performed in a largely consistent manner for almost 100 years using electrosurgical instruments such as an electrosurgical scalpel or resector.

The term “resector” is used in related art to identify a system that includes the set of instruments used for tissue resection (that is, the removal of part or all of a tissue, structure, or organ)—typically, an electrosurgical resection. According to the definition provided, for example, in US 2014/0012077, the resector of related art conventionally includes several functional constituent parts among which there can be found at least an optical system (configured to illuminate the internal tissue to be examined with light from a source of light and to permit imaging of such internal tissue), an active surgical loop (typically a wire-based one, utilized for heat-based ablation of the tissue), a vaporization electrode component, and a translational device (in charge of which is an operator, to enable the spatial positioning and/or orientation of the active surgical loop).

The electrosurgical resector makes use of the heat produced by Joule effect due to the flow of current at radio frequency, for example, through a wire loop. The achieved temperature increase of the wire is a function of power density and time, and when used appropriately, such temperature is made to reach a level sufficient to overheat the tissue and create a coagulation and/or cutting effect. If this apparatus is correctly used, it creates only thermal effects in the tissue, while the electrolytic effects are negligible.

In cutting slithers of prostate with a resistively heated wire loop, urine flow is restored in a procedure coined transurethral resection of the prostate (or, TURP), which is currently the dominant surgical treatment for men suffering LUTS worldwide. While the risk of detrimental effects following TURP is relatively low at about 0.1%, serious lifestyle affecting side effects remain common where up to 10% of men have difficulty achieving or maintaining an erection; short-term but recurrent and long-term postoperative incontinence and retrograde ejaculation are quite common as well. Other risks include urinary strictures, TURP syndrome, urinary tract infections, urine retention, etc.

The use of lasers for ablating or enucleating prostate tissue has gained limited acceptance as an alternative to usage of an electrosurgical instruments and TURP. Having offered the potential for significantly lowering the risks of serious side effects, providing rapid recovery, and even obviating postoperative catheterization and hospitalization, laser-based methods remain expensive and require extensive training to achieve the desired outcomes among a minority of candidate surgeons; few have proved capable of mastering the techniques for a consistent reduction of patient trauma and superior outcomes. It would be novel and useful to devise a laser procedure that required surgical skills, instruments, visualization, and device manipulations substantially identical or very similar to those practiced in TURP, but where reduced risks of TURP syndrome, death, incontinency, and impotence were sustained as consistent outcomes, and where postoperative catheterization and hospitalization were rare and recovery rapid.

Laser enucleation of the prostate with an axially firing fiber (that is, with the use of an optical fiber contraption delivering light output along the axis of the fiber; see, for example, US 2014/0012077, the disclosure of which is incorporated by reference herein) has gained broader use of late for speedier tissue removal and lower cost and potentially reusable laser delivery device, as compared with laser vaporization via single use and costly side firing fibers (that is, the optical fiber contraptions compared to outcouple light substantially transversely to the fiber axis). While a simpler surgery to perform than enucleation, laser vaporization remains significantly slower in eliminating tissue than TURP. The universally recognized failure of the procedure of laser vaporization to maintain superior outcomes as compared to those of the TURP procedure (beyond early experiences by select surgeons) is attributable to manufacturers' efforts to address the relatively low tissue removal rates for laser vaporization in response to comparison with electrosurgical wire loops and allied devices.

While laser generator manufacturers have increased the laser output powers available for clinical trials from the 60 W to 80 W to over 200 W, they largely failed to provide the concomitant increase in side-fire fiber emission geometry that would be required to maintain optimum radiant intensity/irradiance at the chosen target. These developments understandably inevitably resulted in increased likelihood of over-treatment, deep tissue destruction, and surgical side effects while at the same time realizing only modest increase in rates of tissue removal. For example, in one of the initially-offered systems the 80 W Laserscope KTP laser system (that was originally credited with impressive reductions in surgical complications) was paired with the model 2090 side-fire fiber (based upon total internal reflection, or TIR, at a critical angle polished fiber terminus on a 600 μm core fiber). While, upon the increase of the available laser source powers, new 750 μm core side-fire was paired with the third-generation laser at 180 W, in an alternative case of the use of the second-generation laser source at 120 W the same 600 μm core fiber remained the sole offering. The practical situation remains even more egregious for thulium and diode lasers, where side-fire fibers projecting nearly identical radiant intensities (550 μm core and 660 μm core TIR fibers) are used at laser powers in the range from 100 W to more than 200 W.

A skilled person readily appreciates that the ability of a designer to control opto-geometrical parameters of an optical flux formed by the light output from any type of an optical fiber device—and, in particular, the side-fire optical fiber device—is substantially limited. For the side-fire fibers, the maximum diameter and divergence of the light output beam are constricted by the ever-present trade-off between or among competing practical needs and corresponding structural configurations.

For example, the closer the lateral direction of laser light output from the fiber-based contraption is to fully orthogonal (relative to the fiber longitudinal axis), the better the preservation of a circular beam profile, thereby requiring the use of a low fiber numerical aperture. At the same time, the low numerical aperture results in lower degree of beam divergence within the side-fire fiber protective cap, thereby minimizing the diameter of the output beam and maximizing its radiant intensity. Conversely, where higher numerical aperture is employed, the off-axis emission of light is necessarily centered around an axis forming an angle smaller than 90° with the axis of the fiber, and the output light profile becomes elliptical with reduced radiant intensity for the distal side of the ellipse (with the use of such an output beam, more tissue is irradiated but at the expense of greater variation in radiant intensities throughout the output elliptical cross-section of the output beam).

Design options are also limited by purely structural, physical considerations. A person of ordinary skill in the art will readily appreciate that for a laser fiber to be compatible with essentially all working channels of conventionally used cystoscopes, for example, the total diameter of any structure needing to pass through those channels cannot exceed about 1.8 mm. For compatibility with “most scopes” the upper limit of such diameter is about 2.15 mm. Likewise, the length of any rigid section of the fiber-based device is limited to about 14 mm due to the necessity of negotiating a roughly 15° to 30° turn(s) in most scope working channels. Furthermore, options for material of construction to be used for construction of the side-fire fiber-based devices are similarly limited by a necessity for biocompatibility, resistance to very high temperatures, transparency to practically-usable laser light at available wavelengths, i.e., little freedom in selecting among glasses for refractive index is available.

Moreover, in the case of near infrared (near-IR) lasers such as a thulium laser and a holmium laser (the light of which strongly interacts with water, at 1940 nm and 2080 nm), the effective refractive index of the typical operating environment refractive is that of steam and is virtually identical to the refractive index of air, thereby maximizing refraction and Fresnel reflections of the laser light at output surfaces of the side-fire fiber based device, thereby even further restricting design opportunities for increasing the area of tissue exposed to the output laser light and treated with such light.

### SUMMARY OF THE INVENTION

Embodiments of the invention provide an all-optical-fiber device configured to supply, to a target, laser radiation having characteristics sufficient for ablating a biological tissue with such radiation in a manner that is consistent and competitive with the tissue removal geometry and volume provided by conventional electrocautery resection devices. The desired effect is achieved by configuring a facet of the optical fiber or an internal surface of the fiber's termination cap as a surface providing total-internal-reflection of light substantially at every point of such surface while having normals drawn to immediately adjoining surface portions be not parallel with respect to one another.

In particular, embodiments of the invention provide an article of manufacture that includes an optical fiber; and a terminating surface that is transverse to an axis of the fiber and that includes first and second surface portions. Here, a first normal drawn to the first portion is not parallel to a second normal drawn to the second portion, while the terminating surface is dimensioned to redirect light (delivered to the terminating surface through the optical fiber) to form a spatial distribution of so-redirected light outside the terminating surface that is spatially-uninterrupted and arcuate in a plane transverse to the axis of the fiber while, at the same time, not being circumferential about the axis. Substantially in every implementation, the article may be dimensioned to satisfy one or more of the following conditions: (i) a terminating surface is configured to reflect totally-internally the light, delivered to the terminating surface through a dielectric material bound by the terminating surface through the dielectric material; (ii) each of the first and second portions is configured as a substantially planar surface portion while the first and second surface portions have perimeters sharing a perimeter portion; and (iii) the terminating surface is transverse to the axis of the fiber at each and every point of the terminating surface.

Additionally or in the alternative, and substantially in every implementation of the idea of the invention, the article of manufacture may be configured to form the spatial distribution of output light outside the article that subtends (in a plane transverse to the axis of the fiber) an angle smaller than 180 degrees as viewed from the axis of the fiber. Alternatively or in addition, an output end of the optical fiber may be affixed in a tubular hollow of an optical fiber termination element (which forms a part of the article of manufacture), such terminating surface may be made integrally and/or monolithically connected to a wall of the optical fiber termination element.

Embodiments of the invention additionally provide a method for transmitting light through an article of manufacture that includes an optical fiber having a fiber axis. The method includes traversing laser light through a terminating surface (of such article) extending along an axis that is substantially transverse to the fiber axis to form an outcoupled beam of light (here, the laser light is delivered to the terminating surface through the optical fiber) and propagating the outcoupled beam of light outside of the article to form such a focal distribution thereof that is limited in a radial direction by a first curved surface and a second curved surface. The second curved surface is separated radially from the first curved surface and is intersecting the first surface at first and second curves, while the first and second curves are separated from one another along the fiber axis. (A cross-section of such focal distribution in a plane transverse to the fiber axis is arcuate while, at the same time, not circumferential about the fiber axis.) Substantially every embodiment of the method may be configured to form the focal distribution that subtends an angle not exceeding 180 degrees as viewed from the fiber axis.

In at least one specific implementation of the method, the step of traversing includes reflecting the laser light at a surface of an optical fiber termination element and transmitting said laser light through a wall of the optical fiber termination element, wherein said terminating surface is transverse to the fiber axis and internal to the optical fiber termination element, wherein said optical fiber element is affixed inside a tubular hollow of the optical fiber termination element. Optionally, such embodiment of the method may also include a step of transmitting the laser light emanating from the optical fiber element through a lens element (which is located within the optical fiber terminal element between an output facer of the optical fiber element and the terminating surface.

### BRIEF DESCRIPTIONS OF THE DRAWINGS

The invention will be more fully understood by referring to the following Detailed Description of Specific Embodiments in conjunction with the Drawings, of which:

FIG. 1A is a perspective view of a conventionally-used electrocautery cutting accessory for a resection device.

FIG. 1B is a perspective view of another conventionally-used electrocautery cutting accessory for a resection device.

FIG. 1C is a perspective view of a fiber-optic-based cautery accessory for an endovenous device of related art.

FIG. 2 is a perspective view of a side-firing fiber vaporization accessory utilized by a laser resection methodology of related art.

FIG. 3 is a perspective view of a portion of an embodiment of a vaporization/ablation/resection system of the invention configured to form a vaporization/ablation/resection channel in a target tissue, which channel is that substantially equivalent to that cut or formed by a prior art electrocautery device.

FIGS. 4A, 4B, 4C, and 4D are different views of an embodiment of an optical fiber element utilized by the embodiment of FIG. 3. The optional lens element formed at such embodiment at an input surface thereof is illustrated in a dashed line, showing a multiplicity of FIGS. 5A, 5B, and 5C illustrates an optical component configured to be used (within or independently from the optical fiber termination) as an external addition to the optical fiber element, and having the terminating surface configured in a fashion similar to that of the terminating surface of the embodiment of FIGS. 4A-4D. FIGS. 5A-5C additionally illustrate propagation of various output rays of light exiting the terminating surface and formed by light propagating through the embodiment from the input surface of the optical component that, in practice, is positioned to be separated from the output facet of the optical fiber by a gap.

FIGS. 6A, 6B, 6C, 6D, 6E, 6F, 6G, and 6H are various views of alternative/related embodiments of the optical element of the system configured according to the idea of the invention to be used as an optical element external to the output facet of the optical fiber (and, optionally, internal to the optical fiber termination with which the optical fiber is equipped).

FIGS. 7A, 7B, 7C, and 7D offer four views of one embodiment of the invention producing, in operation, a wide, short arc-like shaped off axis light output.

FIGS. 8A, 8B, 8C, and 8D offer four views of a related embodiment of the invention producing, in operation, a narrow, long arc-like shaped off axis light output.

FIGS. 9A, 9B, and 9C show, in a sequence, an application of a typical electrocautery resection loop of related art and a typical tissue lesion produced as a result of such application.

FIGS. 10A, 10B, 10C illustrates, for comparison purposes with the results illustrated in FIGS. 9A through 9C, a typical tissue lesion produced with an embodiment of a laser resection device configured according to the ide 9A-9C of the present invention.

FIGS. 11A, 11B, 11C and 12A, 12B, 12C illustrate related embodiments of laser resection devices in several views.

FIGS. 13A, 13B, 13C, and 13D illustrate an embodiment with a terminating surface structured in a fashion similar to that of embodiment of FIGS. 4A-4D, but with a different number of individual substantially planar surface portion of the terminating surface.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another. While specific embodiments are illustrated in the figures with the understanding that the disclosure is intended to be illustrative, these specific embodiments are not intended to limit the scope of invention implementations of which are described and illustrated herein.

### DETAILED DESCRIPTION

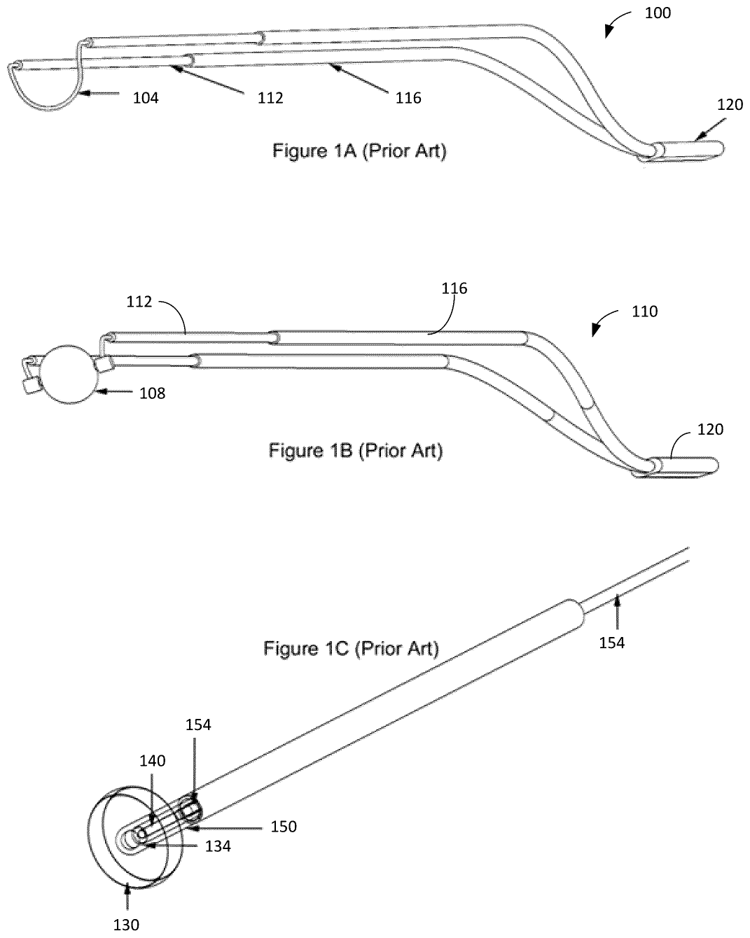

FIGS. 1A and 1B illustrate a conventional accessory 100 to device known as a resector and is used in some endourological procedures (such as benign hypertrophic prostate resection or the removal of vesical neoplasia, for example). The accessory 100 includes an electrosurgical wire loop 104 intended for cutting strips of tissue away from the bulk prostate in TURP procedures, for example (where the loose tissue strips are tucked into the bladder for later morcellation and flushing out). Where excessive bleeding may occur, the electrically-conducting ball 108 (shown in a related embodiment 110 of FIG. 1B) may be employed and used to coagulate the bleeder and may also be used for deep coagulation of tissue (although this practice can have some known problematic consequences). In either case, the active electrode (104, 108) is energized by insulated conductors 112 disposed within the corresponding electrode shafts 114 that lead to the handle 120 and, through it, to carious connections to the electrocautery generator and other components not shown.

FIG. 1C illustrates an embodiment of a laser device (discussed in U.S. Pat. No. 10,092,356, the disclosure of which is incorporated herein by reference) that some have proposed may have utility in laser versions of TURP procedures for mimicking the hemispherical cross-section cuts via direct tissue vaporization. The light output 130 from such a device forms a circumferentially extending over 360° light distribution and is delivered by and outputted from a hollow conical optical element 134. The cone angle is defined relative to the longitudinal axis of the optical fiber 140 and dimensioned for total internal reflection (TIR). The hollow conical optical element 134 is fused within a protective quartz cap 150 and is separate from the optical energy delivery fiber 140, which is stripped of its jacketing layer 154 within the distal portion of the quartz protective cap 150 in order to remove laser labile materials from the vicinity of the location(s) where Fresnel reflections from the fiber's output surface and the optical element input surface occur in operation of the device.

While the device of related art depicted in FIG. 1C may offer acceptable and familiar surgical results to electrocautery devices in for the purposes of TURP with the use of half of the circumferential output 130 (the approximately upper half of distribution 130 that remains above the surface of the tissue), the distribution 130 leads to, at best, wasted energy, and at worst, could damage not targeted tissues including the neurovascular bundle that is known to be important to control urine flow and erectile function.

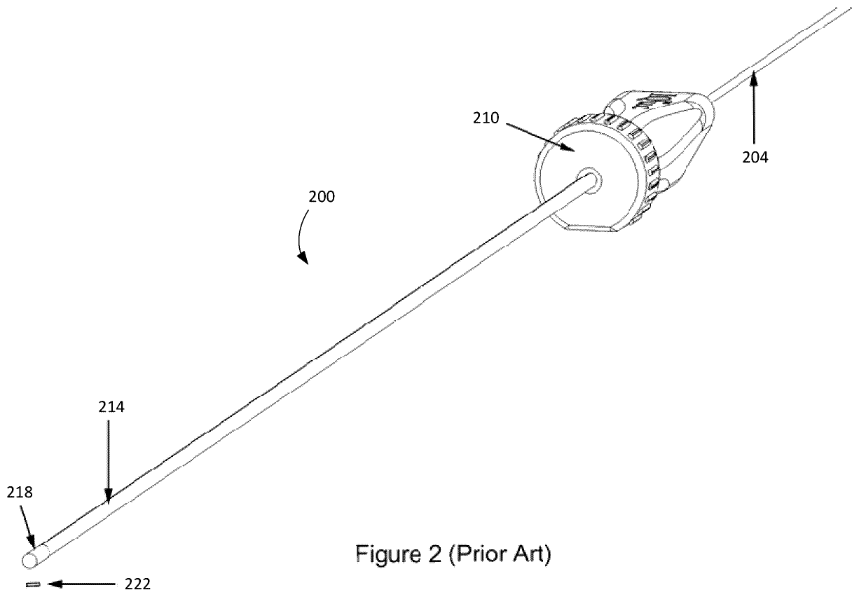

As such, more targeted, pointed approaches for delivering laser energy via bent, axially firing fibers (see, for example, U.S. Pat. No. 5,416,878) or laterally (side) firing fibers for vaporizing prostate tissue (see U.S. Pat. No. 5,486,171) were proposed, or such a device as device 200 depicted in FIG. 2 where the optical fiber 204 is controlled in rotation outside the resectoscope by means of an orientation-and-control knob 210. The knob 210 is connected to the fiber and a protective tube 214 carrying the fiber, which facilitates smooth insertion and removal of the device from the target and secure connection to the device's output cap 218, typically made of fused quartz. This device 200 is structured to emit output laser radiation 222 typically at an angle between 70° and 80° with respect to the longitudinal axis of the fiber 204. Each of these vaporization devices require far more sweeping motions of the tip of the device to be performed for ablating volumes of tissue similar to those as those removed in TURP and, like TURP, laser vaporization with a side firing fiber tends to detrimentally coagulate non-targeted tissues thereby leading to plethora of well-recognized intraoperative and post-operative side effects and complications.

Both a device employing a wire loop and that employing a conventionally structured side-fire fiber have a substantial operational shortcoming in that the laser-light outputs produced by these devices coagulate the target tissue deeper than the exposed tissue surfaces: the wire loop—via the excessive heat required to “melt” the tissue, and the side-fire fiber—via scattering of light from charred portions of the tissue produced by the presence of radiant intensities far exceeding the tissue vaporization threshold.

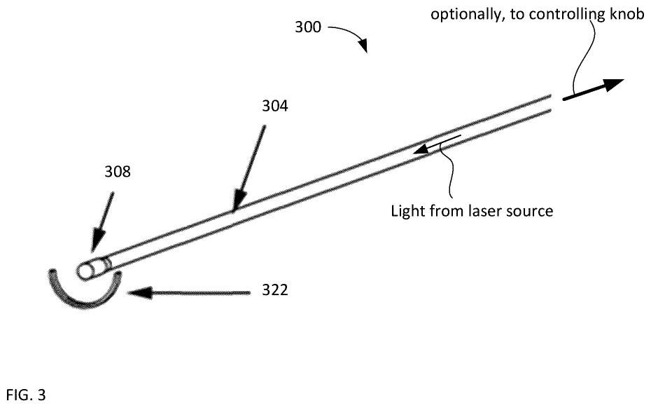

The idea of the present invention stems from the realization that a laser light output from a side-fire optical fiber-based device, shaped in a judiciously defined and discussed below arcual fashion, can be employed to mimic operationally the well-established and familiar electrocautery loop of FIG. 1A. Unlike the existing side-fire optical fibers, embodiments of the invention are equipped with novel elements precisely structured to particularly shape spatial distribution of output light to ensure (among other opto-geometrical characteristics) the TIR at an angle that is greater that characterizing conventional side-fire optical fibers and possess the dimensions that do not result in a fully circumferential light output distribution (in advantageous contradistinction with the currently employed side-fire or radially-emitting optical fibers). A distal end of one embodiment 300 of the proposed device is schematically illustrated in FIG. 3 that depicts the optical fiber inside a protective tube 304 (with the free end of the optical fiber preferably protected by an optical fiber termination, which may be formatted as a fused quartz output cap 308 as discussed below in more detail). The spatial orientation (particularly, a degree of rotation about the axis) of the optical-fiber based device may be optionally controlled with the use of a controlling knob such as knob 210 or such as that disclosed in the U.S. Provisional Patent Application No. 63/405,984 and/or the U.S. Design patent application Ser. No. 29/853,120 (the disclosure of each of which is incorporated herein by reference). The spatial distribution of outcoupled light (which is arcuate in a plane transverse to the fiber axis while, at the same time, not circumferential about the axis) is denoted in FIG. 3 as 322.

Conventionally—and as currently employed in related art—laser light is redirected in the side-fire fibers with the use of a highly polished surface that is set at an angle defined as “critical” to effectuate total internal reflection according to Snell's law, and usually formed at the fiber itself. It is preferable to redirect the laser light at a right angle to the fiber's longitudinal axis, but—while Snell's law typically yields around 46° which is more than sufficient for right angle deflection—the practically achievable maximum value of the fiber's facet angle is set by a need to accommodate and include into the fiber output all fiber modes propagating within the fiber at different angles—those approaching the TIR plane both above and below the meridional or longitudinal ray. Because the most common fiber used with near-IR lasers has the numerical aperture (NA) of 0.22 NA and the fused silica core with a refractive index of about 1.457, one must reduce the calculated TIR angle maximum by a coefficient representing the arcsine of the numerical aperture divided by the ratio of the fiber core refractive index and the external medium, typically air (nₐᵢᵣ=1.000)—to about 37°.

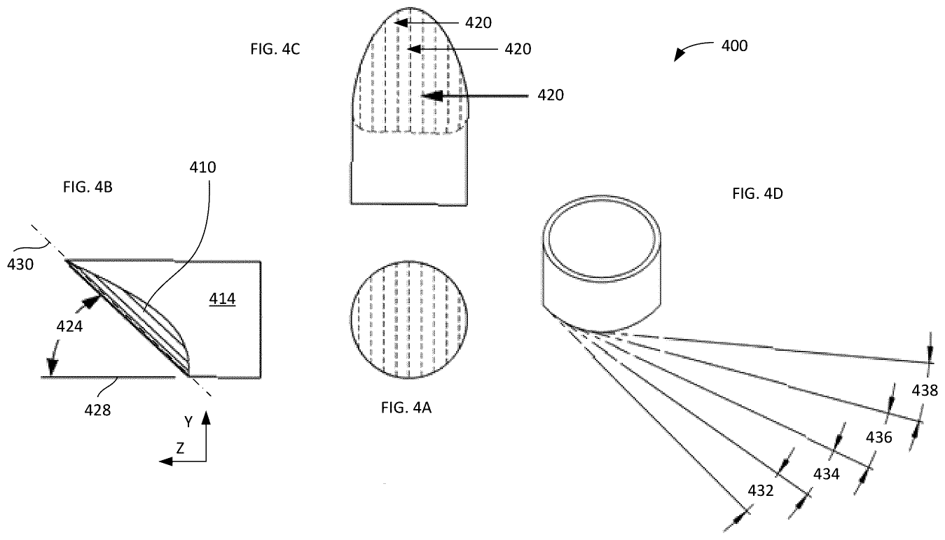

FIGS. 4A, 4B, 4C, and 4D schematically illustrate, in several views, an embodiment 400 of the output portion of the optical-fiber based device; the embodiment 400 contains a TIR-causing terminating surface 410. The surface 410 is judiciously structured and dimensioned to so totally-internally-reflect light propagating in the optical fiber 414 and incident onto this surface as to form, outside of the embodiment 400, a light output distributed along a spatial arc (˜a substantially arcuate light output distribution, indicated in FIG. 3 as 322) rather than the elliptical spot resulting at 74° off axis as in most side-fire fibers of related art. As shown, the surface 410 is formed by multiple surface portions 420 (shown in this specific example, for simplicity, as substantially planar surface portions) generated across the fiber core and sharing portions of their boundaries or perimeters. Each of these surface portions is inclined at the TIR angle 424 with respect to axis 428 (which axis is parallel to the axis of the optical fiber) and thus extends along the local axis 430 that is inclined at the angle 424 with respect to the axis 428. At the same time, however, the immediately neighboring surface portions 420 form a dihedral angle of X° with one another (in each pair of the neighboring surface portions 420) as is schematically indicated by 432, 434, 436, 438 in FIG. 4D. Notably, the embodiment 400 may be implemented by simply appropriately structuring the output end of the optical fiber, or as an addition to a stand-alone optical fiber that is juxtaposed (permanently or in a separable fashion) with a facet of such optical fiber.

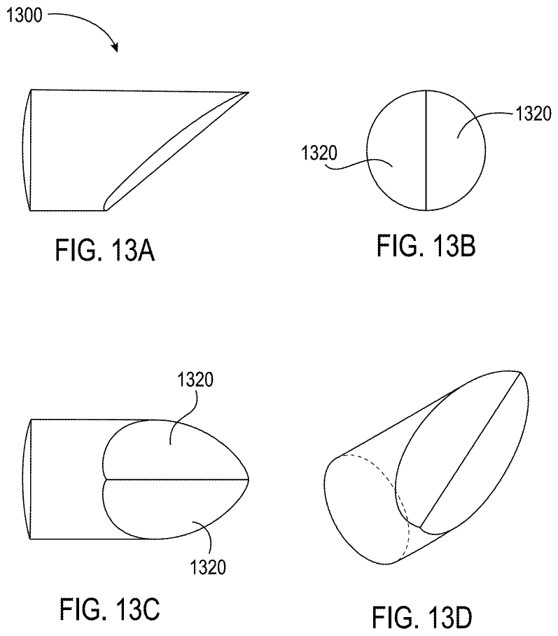

In practice, the overall light output will be formed by multiple constituent output sub-beams of light each of this emanates through a respectively corresponding surface portion 420. For ease of appreciation of the principle of operation of this embodiment, a version 1300 in which there are only two constituent surface portions 1320 (which functionally correspond to the surface portions 420 of the embodiment 400) is additionally sketched in different views in FIGS. 13A, 13B, 13C, and 13D. Notably, as a person of skill will readily appreciate, the surface opposite to the two-facet surface is shown to be structured as a convex surface to form a positive optical lens element. Here, light propagating along the axis through the embodiment 1300 from the convex surface to the two-facet surface exits the two-facet surface in the form of two output beams that are inclined with respect to one another.

Where X° is the same for each immediately-adjacent pair of portions 420, the total angle subtended by the arc of the light output 322 is substantially twice the product of X and the number N of surface portions 420 of the surface 410, less 1, plus the full angle of divergence of light required for the fill within the fiber at the TIR. The full angle of divergence is schematically indicated in FIGS. 5A, 5B and 5C. FIGS. 5A through 5C illustrate an embodiment 500 possessing the same surface 410 as that of the embodiment 400. Notably, the embodiment 500 differs from the embodiment 400 in that embodiment 500 is configured as an add-on termination component, the input surface 520 of which is shaped to form a lens element, to receive and couple into the component 500 the freely propagating beam of light 510.

In one example, assuming the surface 410 is composed of ten surface portions 420 with a dihedral angle X°=5° between each of the two immediately-neighboring each other surface portions, a light mode filled fiber element 400, 500 with the numerical aperture NA would produce the output arc 322 subtending about 115° as seen from the fiber axis.

Spatial smoothing of the profile of the beam of light emerging through the surface 410 may be optionally accomplished in the plane substantially orthogonal to the fiber's longitudinal axis via altering the angles between the surface portions 420 (that is, in the example of FIG. 4D, by ensuring that 432≠434≠436≠438, and/or by varying the widths of the individual surface portions 420 to select which portions of the beam profile do overlap and how much they overlap.

While a corresponding multi-faceted surface of each of the embodiments 400, 500, and 1300 is shown to be structured in a convex fashion (when such surface forms sort of a dome above the peripheral ends of this surface as seen from outside of a given embodiment), it is appreciated that in a related embodiments the multiplicity of facets or surface portions 420, 520, 1320 can be arranged in a concave fashion. For the purposes of the disclosure and appended claims, and when the terminating surface of a given invention is formed as a multi-faceted surface (see 420, 530, 1320 as examples), such surface is defined to be convex if and when every dihedral angle formed by the immediately-neighboring surface portions (that is, facets) of such terminating surface measures less than 180° as seen from a point located on an axis of the embodiment inside the embodiment. On the other hand, such surface is defined to be concave when every dihedral angle so-formed and so-seen measures more than 180°.

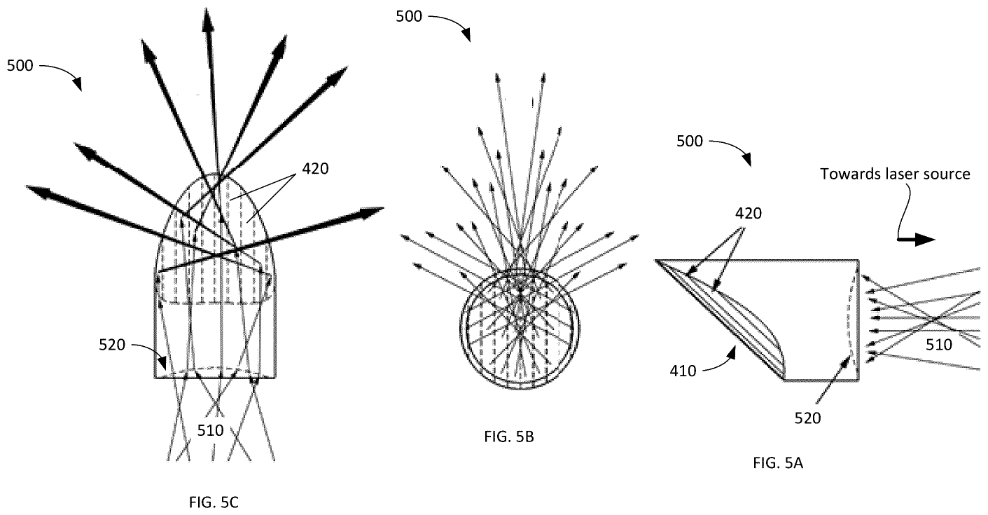

FIGS. 6A, 6B, 6C, 6D, 6E, and 6H illustrate two embodiments 600, 650 that are related to embodiments 400 and 500. However, in comparison with the embodiments 400, 500, the TIR surfaces of the embodiments 600, 650 do not includes multiple substantially planar surface portions but are configured, instead, as spatially substantially monotonic surfaces (defined to be either concave or convex). In particular, FIGS. 6A, 6B, 6C, and 6D illustrate in several views an embodiment 600 possessing a spatially-monotonic TIR surface 610 having a positive curvature and generally inclined with respect to the fiber axis by an angle 614 (as shown by the angle 614 separating the fiber axis and the axis 616. FIGS. 6E, 6F, 6G, and 6H illustrate in several different views an embodiment 650 possessing a spatially-monotonic TIR surface 660 with a negative curvature (the inclination of which to the fiber axis is shown as the angle 654 between the axis 658 and the surface substantially parallel to the fiber axis). Each of the surfaces 610, 660 is differentiable substantially at every point of such surface. The sign of a curvature is defined according to the well-known convention; the center of curvature of the surface 610 in any plane containing the axis of the embodiment 600 lies to the left of the surface 610 while the sign of curvature of the surface 660 in any plane containing the axis of the embodiment 650 lies to the right of the surface 660.

While both of the embodiments 600 and 650 were manufactured and employed, manufacturing the negative curvature half-cone cap 650 in practice proved limited in success due to unintended melting and insufficient quality fusion between the thin walls of the element and the protective quartz cap. The positive curvature half-cone structured according to the embodiment 600 proved readily manufacturable and preferred. It is understood that the TIR surfaces 610, 660 of the embodiments 600, 650 are also, substantially, formed by a multiplicity of substantially planar surface portions (by analogy with the surface 410, for example). However, the dimensions of such constituent substantially planar surface portions are infinitesimally small while any two of such planar surface portions (including the immediately-neighboring and adjoining each other surface portions) that are transverse to a plane in which the curvature of the surface 610, 600 is defined to have corresponding normals that are necessarily tilted with respect to one another (that is, not parallel to one another).

It will be understood by those skilled in the art that a curvature of the TIR surface of an embodiment of the invention in a plane that is substantially parallel to axes shown as 616, 658 may serve to expand the light output substantially spatially-continuously in the plane containing the fiber axis. In practice, such curvature can be readily produced but the practical utility of the result in not particularly justified in that curves with tangent planes at angles higher than those allowed by Snell's law merely refract the laser light in a largely axial direction such that only tangent planes lower than the TIR maximum are functional, e.g., lower than 37° for mode filled 0.22 NA fiber. Accordingly, the presence of this additional curvature serves only to stretch the elliptical distortion already present and to exaggerate the variances in the radiant intensity within the beam profile.

A skilled person will readily understand that divergence of light in the arcual distribution 322 along the axis of the fiber remains defined by the mode fill as would be expected in the case of a standard fiber output divergence. However, in a specific case—that is when the embodiment of the invention is configured as an accessory fused quartz or sapphire rod or cap that is spatially separate from the optical fiber (for reasons stated in U.S. Pat. No. 9,323,005, the disclosure of which is incorporated herein by reference), opportunities arise for modifying the angular and spatial profile of the light distribution exiting the optical fiber and impinging on the input surface of the accessory optical element 400, 500 (aka the power surface) before encountering the multiplanar TIR surface 410 or prior to imparting the smooth, or infinitely multiplanar, TIR surfaces of the related embodiments of FIGS. 6A through 6H. For such a purpose, spatial configuration of the input surfaces of the embodiments 400, 500, 600, 650—that is, surfaces opposite to the TIR surfaces 410, 610, 660—can be appropriately spatially curved to form corresponding lens elements (as shown, for example, by surfaces 520 of FIGS. 5A-5C and 620, 670 of FIGS. 6A-6H.

It should be noted that embodiments such as 400, 500 containing TIR surfaces 410, 510 that include multiple planar constituent surface portions (the multi-planar embodiments) do have functional utility in that the beam divergence of the base or feed fiber (that is, the optical fiber delivering light to the embodiment 400, 500) is broad enough for the user to select the widths and angles of the planes to control overlapping reflections to smooth profiles or purposefully direct much of the energy at targeted angles. In combination with the other available beam shaping surfaces—the fiber input surface as described in U.S. Pat. Nos. 9,122,009 and 9,233,089 (the disclosure of each of which is incorporated by reference herein), the fiber output surface and optical element input surface as described in U.S. Pat. No. 9,488,783 (the disclosure of which is incorporated by reference herein)—a high degree of uniformity may be achieve in the radiant intensity of the arc output, the arc of circumscription of which is controlled by the arc of the TIR planes or curvature as described above.

The utility of such control of the spatial distribution of the resulting light output may be critical for optimization of laser-to-tissue interaction in laser surgery where undertreatment due to insufficient radiant intensity fosters tissue adhering to the protective cap and over treatment due to radiant intensities significantly above the vaporization threshold for the tissue results in charring. Areas of the quartz capsule where tissue adheres rapidly heat as more and more laser energy is absorbed as the tissue degrades, serving to heat adjacent surfaces—those adjacent surfaces where tissue cannot adhere due to the high radiant intensity but where alkali and alkaline earth metal ions and counter ions readily intercalate and initiate a devitrification cascade failure that erodes the output surface and those adjacent surfaces previously spared tissue adhesion for having been insufficiently heated.

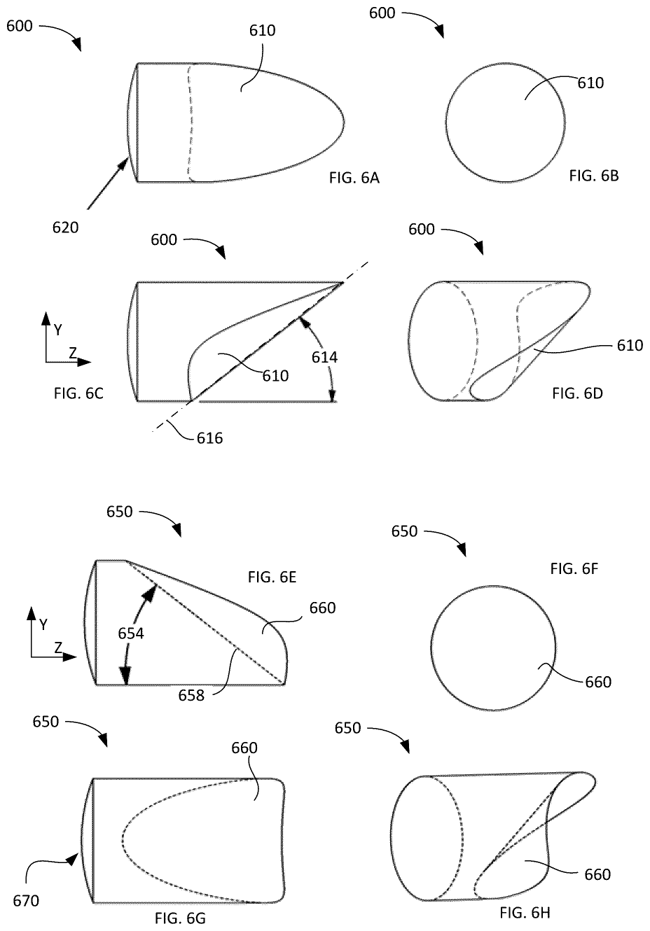

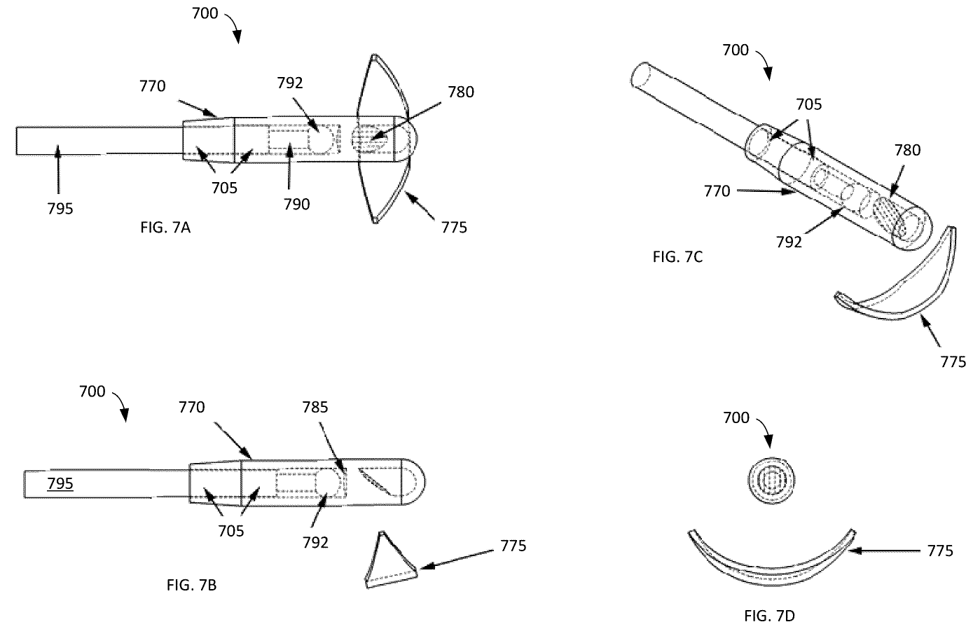

Understandably, the desired irradiance/intensity distribution of the light output for all tissue vaporization applications should be as close to uniform across the output surface as possible, preferably with sharp edges to minimize transition zones subject to tissue adhesion to the optical fiber device. To this end, FIGS. 7A-7D and 8A-8D illustrate but two possible embodiments of the system of the invention (that respectively utilize stand-alone, spatially separate from the optical fiber elements with TIR surfaces configured similarly to the surfaces 410 and/or 510 and/or 610 and/or 660) that, in operation, outcouple light from sideways with respect to the axis of the constituent optical fiber to form arcually-shaped light outputs dimensioned according to the idea of the invention.

In particular, FIGS. 7A-7D illustrate an embodiment 700, in which the optical fiber 795 (with the bare glass fiber body portion 790 exposed but otherwise mostly coated with the polymeric coating) is complemented and cooperated with a generally tubular termination element 770 (configured—in the example shown—as a protective quartz cap having an open input end—into which the optical fiber is inserted to be affixed therein and a closed, sealed end shown in the diagram of FIGS. 7A-7C as a substantially hemispherically shaped end of the terminal 770). The jacket of the fiber 795/790 provides strain relief in the protective quartz cap 770, held onto the fiber with adhesive at 705. Inside the hollow of the termination element 770, the output facet of the fiber 795/790 is cooperated with the ball lens element 792. Light emanating from the optical fiber through the ball lens 792 may made, in at least one specific case, to traverse additionally an auxiliary spatially-curved surface formed inside the termination element 770—such as a convex surface 785—prior to impinging onto the multi-faceted TIR surface 780 that is structured according to the principle discussed above in reference to the surface 410 of the embodiment 400 (or the surface 510 of the embodiment 500). While light that is delivered through the fiber 795/790 towards the termination element 770 traverses the outer surface of the termination element 770 after being totally-internally reflected by the TIR surface 780, the skilled person appreciates that the surface 780 is—functionally speaking—the output surface of the embodiment 700.

In operation, the embodiment 700 produces a short arc (as viewed in the plane transverse to the axis of the fiber 795) the light output distribution 775 that is broadened by increasing the divergence of the fiber 795/790 to the maximum acceptance for the multi-faceted TIR surface 780 by use of the ball lens 792 on the bare fiber 790 terminus and a convex lens 785 on the optical element power surface.

FIG. 8A-8D illustrate an embodiment configured to produce a wide arcually-shaped light output 820 from an optical fiber juxtaposed with a termination element (configured in this specific example as a protective quartz cap) 840 configured to contain, inside the termination 840, an optical element 835 defining a spatially-monotonic TIR surface 825 configured according to the principle discussed in reference to the embodiments 600, 650. Here, by analogy with the embodiment 700, the optical fiber 845 is glued/adhered 850 into/inside the protective quartz cap 840 tubular hollow, into which the optical element 835 is fused. It is worth noting that failure to fuse the optical element 835 into the termination cap 840 will very likely result in considerable Fresnel reflections and even total internal reflections for the light exiting the cylindrical side of the optical element and additional Fresnel reflections upon entering the protective cap inner cylindrical wall. In addition, the presence of a lens surface 860 on the distal tip of the bare fiber body 855 and input or “power” spatially-curved surface 830 of the optical element 835 are enabling an embodiment where the length of the protective cap 840 length must be kept short to pass endoscopic working channels; flat surfaces will produce Fresnel reflections at angles low enough to damage lase labile materials such as adhesive 850 and the fiber polymeric jacket coating.

While light that is delivered through the fiber 855/845 towards the termination element 840 traverses the outer surface of the termination element 840 after being totally-internally reflected by the TIR surface 825, the skilled person appreciates that the surface 825 is—functionally speaking—the output surface of the embodiment 800.

A skilled person now readily understands that—in reference to illustrations provided by FIGS. 7A-7D and 8A-8D—an embodiment of invention, in operation, is configured to traverse laser light (delivered to a terminating surface of the embodiment through an optical fiber) through such terminating surface extending along an axis that is substantially transverse to the fiber axis to thereby form an outcoupled beam of light, and to propagate the outcoupled beam of light outside of the embodiment to form a focal distribution of light that is limited in a radial direction by a first curved surface and a second curved surface. Here, the second curved surface is separated radially from the first curved surface while intersecting the first surface at first and second curves. The first and second curves are separated from one another along the fiber axis. A cross-section of the focal distribution in a plane transverse to the fiber axis is arcuate while, at the same time, not circumferential about the fiber axis.

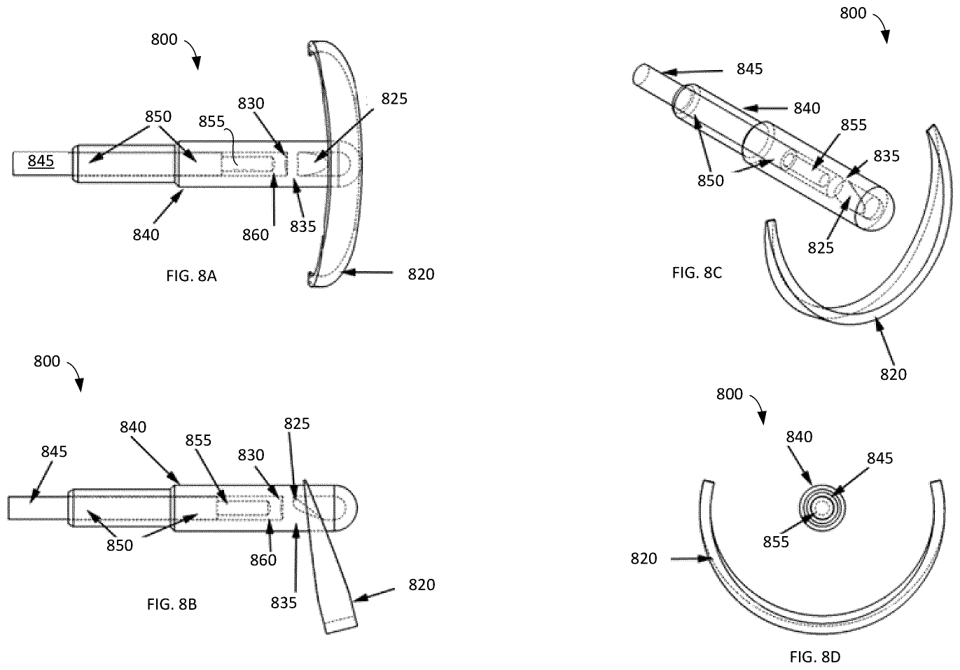

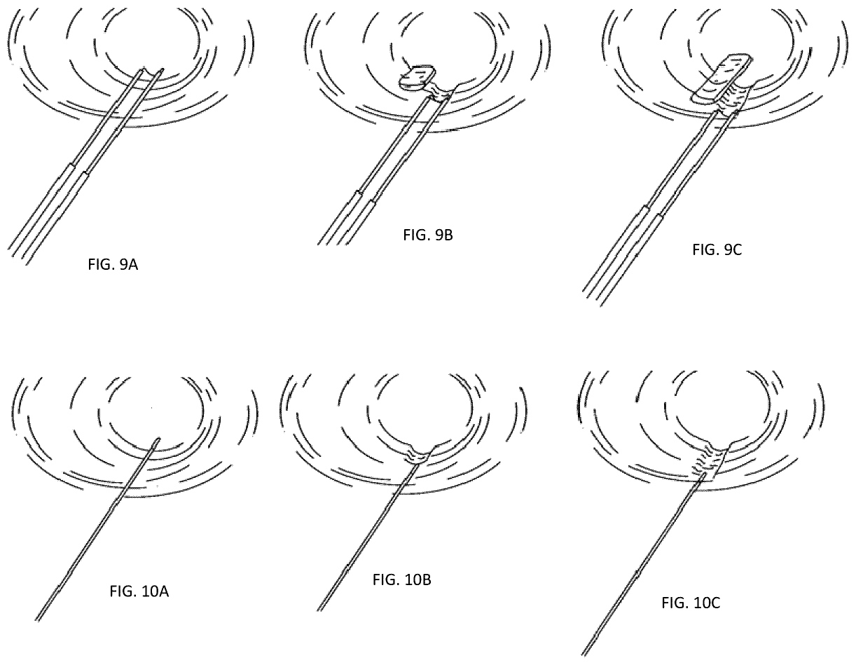

FIGS. 9A, 9B, 9C provide a sequence of illustrations of application of an electrocautery loop of the resector of related art in resection of a strip of prostate tissue. In FIG. 9A, the loop is positioned at the posterior prostate gland edge and current is applied to heat the loop. In FIG. 9B the loop is drawn anteriorly to begin a cut of tissue (poorly illustrated as separated from the bulk gland) and by the time shown in FIG. 9C the loop has been raised through the tissue to terminate the cut. In stark advantageous contradistinction, embodiment embodiments of the invention described herein performs the surgery in precisely the same manner as the electrosurgical loop of related art, but the tissue is vaporized rather than excised. The tip of the device (such as that illustrated in embodiments 700, 800) is placed near the posterior edge of the prostate lobe and the laser is activated, FIG. 10A. The fiber device is simply withdrawn while in contact with the gland (FIG. 10B) until the cut reaches the desired terminus (FIG. 10C).

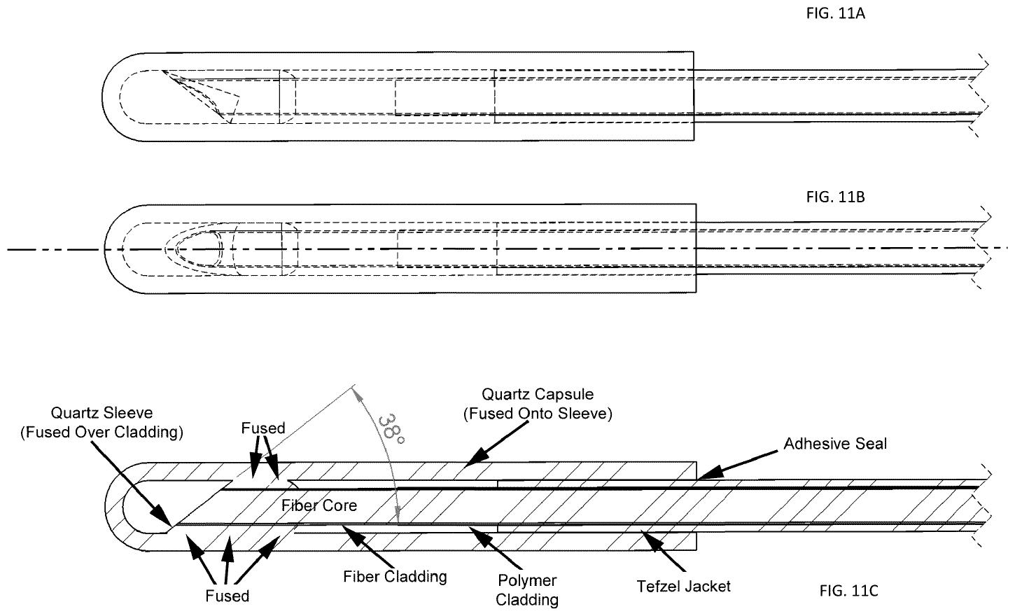

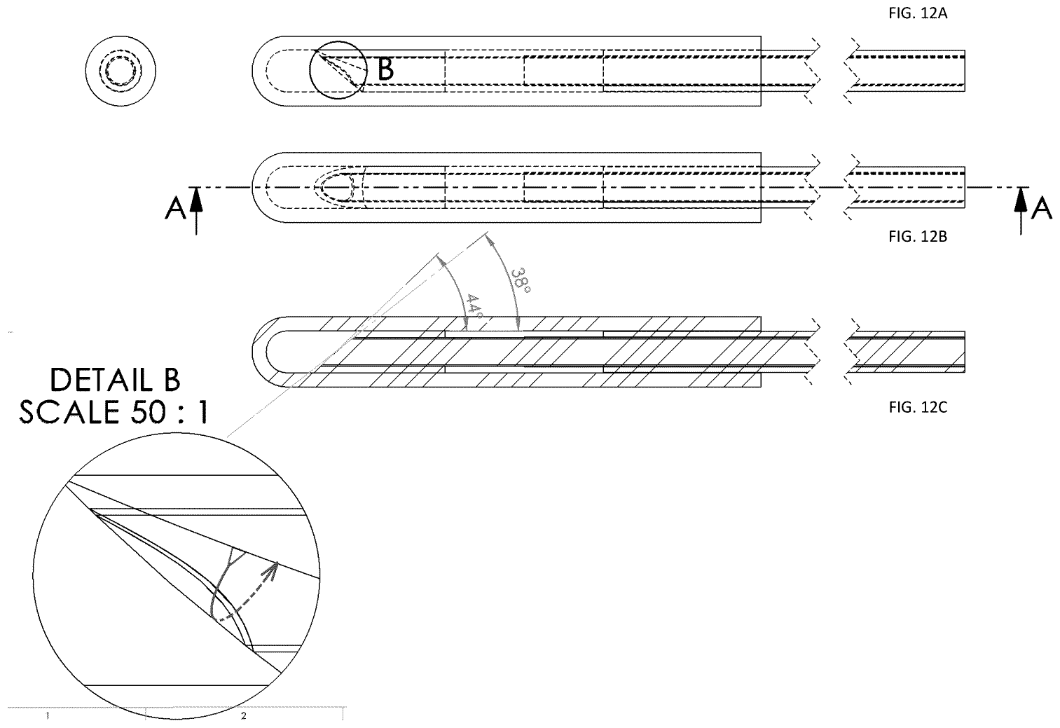

FIGS. 11A, 11B, 11C and 12A, 12B, 12C illustrate related embodiments of laser resection devices in various views. Here, an article of manufacture representing an embodiment of a laser resection device that includes an optical fiber and an optical fiber termination. The embodiment includes a TIR-surface that is spatially separated from the output facet of the optical fiber. Such TIR-surface is transverse to the optical fiber axis and includes multiple surface portions configured, as discussed above, such that a first normal drawn to the first surface portion is not parallel to a second normal drawn to the second surface portion. The TIR-surface is dimensioned to redirect light, delivered to the TIR-surface through the optical fiber, to form a spatial distribution (of so-redirected light outside the TIR surface) that is spatially-uninterrupted and arcuate in a plane transverse to the axis of the fiber while, at the same time, not being circumferential about the fiber axis. The embodiment further comprises an intermediate surface between the output facet of the optical fiber and the TIR-surface, while the output facet and the intermediate surface are separated by a gap along the fiber axis (here, the gap may contain gas but is devoid of a material of the optical fiber and/or the material of the optical fiber termination. As shown in these examples, the output end of the optical fiber is affixed in a tubular hollow of the optical fiber termination element. The TIR-surface may be integrally and/or monolithically connected to a wall of the termination element. Optionally, the embodiment includes at least one curved surface between the output end of the optical fiber and the TIR-surface and, in at least one special case, such at least one curved surface is a surface of a lens element located between an output facet of the optical fiber and the TIR-surface.

Overall, as a skilled person will readily appreciate, embodiments of the discussed resection device produce the desired effect by employing light only (and would be characterized as all-optical-fiber resection devices) in are structured to be compatible with laser sources producing up to a kW of continuous or time-averaged pulsed optical power in either ultraviolet, visible and/or near-infrared spectral regions and cystoscopes/endoscopes used to provide minimally invasive access and visualization/translation during tissue resection. Embodiments may be employed for resection and/or ablation and/or vaporization and/or haemostasias via manipulations consistent with those employed using a traditional electrosurgical loop-based resection devices. As described, in operation the embodiments provide a means of controlling the amount/extent of tissue irradiated by a laterally emitting optical fiber device, particularly where such control encompassed a range of fiber output parameters compatible with maintaining optimum radiant intensity for a broad range of laser powers exceeding 80 W and ideally up to as much as an order of magnitude higher, e.g., ˜10× larger area of tissue irradiation. Particular advantage of utilizing the embodiment of the invention comes from employing these embodiments with smaller core fibers as opposed to larger core optical fibers, i.e., in a situation when lateral output beam characteristics are largely independent of the diameter of the fiber core.

The disclosure of each of patent documents or other documents referred to in this application is incorporated herein by reference.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself. The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The term “A and/or B” or a similar term means “A alone, B alone, or A and B together” and is defined to be interchangeable with the term “at least one of A and B.”

While the invention is described through the above-described exemplary embodiments, it will be understood by those of ordinary skill in the art that modifications to, and variations of, the illustrated embodiments may be made without departing from the inventive concepts disclosed herein. Disclosed aspects, or portions of these aspects, may be combined in ways not listed above. Accordingly, the invention should not be viewed as being limited to the disclosed embodiment(s).

1. An article of manufacture comprising:
an optical fiber; and
a terminating surface that is transverse to an axis of the fiber and that includes first and second surface portions,
wherein a first normal drawn to the first portion is not parallel to a second normal drawn to the second portion,
wherein said terminating surface is dimensioned to redirect light, delivered to said terminating surface through the optical fiber, to form a spatial distribution of so-redirected light outside the terminating surface that is spatially-uninterrupted and arcuate in a plane transverse to the axis of the fiber while, at the same time, not being circumferential about the axis.

2. An article of manufacture according to claim 1,
(2A) wherein said terminating surface is configured to reflect totally-internally said light, delivered to the terminating surface through a dielectric material bound by the terminating surface through said dielectric material; and/or
(2B) wherein each of the first and second portions is configured as a substantially planar surface portion,
wherein the first and second surface portions have perimeters sharing a perimeter portion; and/or
(2C) wherein the terminating surface is transverse to the axis of the fiber at each and every point of the terminating surface; and/or
(2D) wherein the terminating surface is a convex surface

3. An article of manufacture according to claim 1, wherein the first normal is in a first plane, the second normal is in a second plane, the first and second planes being substantially parallel to one another.

4. An article of manufacture according to claim 3, wherein each of said first and second planes is substantially transverse to the axis of the fiber.

5. An article of manufacture according to claim 1, wherein the terminating surface includes a third surface portion such that a third normal drawn to a surface of the third portion is not parallel to each of the first normal and the second normal.

6. An article of manufacture according to claim 1, wherein said terminating surface defines a spatially-monotonically convex surface or wherein said terminating surface defined a spatially-monotonically concave surface.

7. An article of manufacture according to claim 1, wherein said terminating surface intersects third and fourth planes along respective first and second curved lines, wherein the third and fourth planes are transverse to one another and substantially parallel to the axis of the fiber.

8. An article of manufacture according to claim 1, wherein said terminating surface is a surface of an output facet of the optical fiber or wherein said terminating surface is a surface spatially separated from the output facet of the optical fiber along the axis of the fiber.

9. An article of manufacture according to claim 8, wherein, when the terminating surface is a surface spatially separated from the output facet of the optical fiber, the article further comprising an intermediate surface between the output facet of the optical fiber at the terminating surface, said output facet and said intermediate surface separated by a gap along the axis of the fiber.

10. An article of manufacture according to claim 1, wherein an output end of the optical fiber is affixed in a tubular hollow of an optical fiber termination element, and wherein said terminating surface is integrally and/or monolithically connected to a wall of the optical fiber termination element.

11. An article of manufacture according to claim 10, comprising at least one curved surface between the output end of the optical fiber and said terminating surface.

12. An article of manufacture according to claim 11, wherein said at least one curved surface is a surface of a lens element disposed between an output facet of the optical fiber and said terminating surface.

13. An article of manufacture according to claim 1, wherein said spatial distribution formed outside the surface subtends an angle that is smaller than 180 degrees as viewed from the axis of the fiber.

14. An article of manufacture according to claim 1, wherein the terminating surface is a concave surface.

15. A method comprising:
transmitting light through an article of manufacture that includes an optical fiber having a fiber axis by:
traversing laser light through a terminating surface of said article extending along an axis that is substantially transverse to the fiber axis to form an outcoupled beam of light, wherein said laser light is delivered to said terminating surface through the optical fiber; and
propagating the outcoupled beam of light outside of the article to form a focal distribution thereof, wherein the focal distribution is limited in a radial direction by a first curved surface and a second curved surface, the second curved surface being separated radially from the first curved surface, the second curved surface intersecting the first surface at first and second curves, the first and second curves being separated from one another along the fiber axis,
wherein a cross-section of said focal distribution in a plane transverse to the fiber axis is arcuate while, at the same time, not circumferential about the fiber axis.

16. A method according to claim 15, wherein said focal distribution subtends an angle not exceeding 180 degrees as viewed from the fiber axis.

17. A method according to claim 15, wherein said propagating includes transmitting the laser light, delivered through the optical fiber element, through a first surface portion and a second surface portion of said terminating surface,
wherein a first normal drawn to the first surface portion and a second normal drawn to the second surface portion are not parallel to one another.

18. A method according to claim 15, wherein the first and second surface portions adjoining one another to share a portion of perimeters thereof and defining a dihedral angle therebetween.

19. A method according to claim 15,
wherein said propagating includes transmitting the laser light, delivered through the optical fiber element, through the first and second surface portions each of which is configured as a substantially planar surface portion.

20. A method according to claim 15, further comprising outcoupling said laser light from the optical fiber and changing a degree of divergence of said laser light upon propagation thereof from the optical fiber to said terminating surface.

21. A method according to claim 20, comprising propagating said laser light through a gap and/or through a curved surface configured as a surface of a lens element.

22. A method according to claim 15, wherein said traversing includes reflecting said laser light at a surface of an optical fiber termination element and transmitting said laser light through a wall of the optical fiber termination element, wherein said terminating surface is transverse to the fiber axis and internal to the optical fiber termination element, wherein said optical fiber element is affixed inside a tubular hollow of the optical fiber termination element.

23. A method according to claim 22, further comprising transmitting said laser light emanating from the optical fiber element through a lens element that is located within the optical fiber terminal element between an output facer of the optical fiber element and said terminating surface.
